(19) United States
(12) Patent Application Publication
(10) Pub. No.: US 2022/0135499 A1
(43) Pub. Date: May 5, 2022

(54) OLEFIN ISOMERIZATION WITH SMALL CRYSTALLITE ZEOLITE CATALYST

(71) Applicant: Lyondell Chemical Technology, L.P., Houston, TX (US)

(72) Inventors: Rick B. Watson, Houston, TX (US); David W. Leyshon, Houston, TX (US)

(73) Assignee: Lyondell Chemical Technology, L.P., Houston, TX (US)

(21) Appl. No.: 17/519,862

(22) Filed: Nov. 5, 2021

Related U.S. Application Data
(60) Provisional application No. 63/110,178, filed on Nov. 5, 2020.

(51) Int. Cl. C07C 5/27; B01J 29/65; B01J 35/02; B01J 37/00

(52) U.S. Cl. CPC C07C 5/2708; B01J 29/65; C07B 2200/09; B01J 37/0009; C07C 2529/65; B01J 35/023

(57) ABSTRACT

A skeletal isomerization process for isomerizing olefins is described. The process includes the steps of feeding an olefin-containing feed to a reactor having an isomerization catalyst with a small crystalline size that is less than 1 μm in all directions. The small crystalline size increases the life of the catalyst and the yield of skeletal isomer products, as well as reducing the formation of heavy C5+ olefin byproducts, as compared to processes using conventional catalyst with crystalline sizes of 1 μm or more.

### PRIOR RELATED APPLICATIONS

This application claims the benefit of priority to U.S. Provisional Patent Application No. 63/110,178, filed on Nov. 5, 2020, which is incorporated herein by reference in its entirely.

### FEDERALLY SPONSORED RESEARCH STATEMENT

Not applicable.

### FIELD OF THE DISCLOSURE

The disclosure generally relates to skeletal isomerization processes, and more specifically to a method of improving the performance of an olefin skeletal isomerization process.

### BACKGROUND OF THE DISCLOSURE

Zeolite materials, both natural and synthetic, are known to have catalytic properties for many industrially relevant chemical reactions. Zeolites are ordered porous crystalline aluminosilicates having a definite structure with cavities interconnected by channels. The cavities and channels throughout the crystalline material can be of such a size to allow selective reaction of hydrocarbons. Such hydrocarbon reactions by the crystalline aluminosilicates essentially depends on discrimination between molecular dimensions. Consequently, these materials in many instances are known in the art as “molecular sieves” and are used, in addition to catalytic properties, for certain selective adsorptive processes.

In many instances, it is desirable to convert a methyl branched olefin such as isobutylene, to a linear olefin, such as 1-butene, by mechanisms such as skeletal isomerization. It is known, by the demand for Patent EP 0523 838 (Lyondell), that it is possible to use a process of skeletal isomerization of linear olefins, or iso-olefins, with a catalyst of zeolite type for converting the linear olefins to iso-olefins, or vice versa.

Up until now, catalysts for the skeletal isomerization of olefins, particularly from isobutylene to butene or from butene to isobutylene, have utilized large crystal zeolites (≥1 micron) together with an associated catalytic metal such as platinum, palladium, boron or gallium. Such zeolites suffer from short cycle lengths (about 7 to 10 days) due to deactivation by coking, which require frequent regeneration or performance altering conditions such as dilution or lower temperature/pressure operation.

Thus, there exists a need for improvements to the skeletal isomerization process and/or isomerization catalysts to increase the catalyst cycle before regeneration. Ideally, such improvements will also result in increases in yield of desired products.

### SUMMARY OF THE DISCLOSURE

The present disclosure is directed to novel methods for structurally isomerizing hydrocarbon streams containing one or more olefins. In particular, a skeletal isomerization process that includes a zeolite catalyst with a smaller crystallite size than conventional isomerization catalysts, and an increase feed flow rate and/or a decreased reactor temperature is disclosed.

The smaller crystallite size (<1 μm in all directions) was found to be a more active catalyst compared to conventional catalyst (≥1 μm in diameter) when the same feed rate was used. This means less of the smaller crystallite size catalyst will be needed compared to the conventionally sized zeolite catalysts, which reduces the costs of the skeletal isomerization process.

Because of the increase in activity, the feed flow can be increased, or a combination of an increase in feed flow and a decrease in reactor temperature, can occur without decreasing the yield of desired products. An increase in the conversion of reactant olefins to product olefins, as well as reductions in the production of heavy byproducts, herein referred to as “C5+ heavies”, were obtained at the higher feed flow by itself, or in combination with lower reactor temperature. Further, a longer catalyst cycle compared to processes using conventionally sized zeolite catalysts also occurs.

Some aspects of the presently disclosed method comprise the steps of providing a feed comprising one or more olefin(s) to a reactor containing a zeolite catalyst with a small crystallite, wherein the reactor is maintained at a first temperature. The one or more olefin in the feed is structurally isomerized to at least one skeletal isomer in the reactor. The use of the small crystallite catalyst extends the catalyst cycle by at least 30% compared to processes using catalysts with conventionally sized crystallites.

In other aspects of the presently disclosed method, the method comprises the steps of providing a feed comprising one or more olefin to a reactor containing a zeolite catalyst with a small crystallite, wherein the reactor is maintained at a first temperature. The feed is provided at a weight hourly space velocity (WHSV) that is at least three times as fast as the WHSV that is used with conventionally sized zeolite catalyst. By changing the catalyst crystalline size and increasing the feed flow, the catalyst cycle is extended by at least 30% compared to processes using catalyst with conventionally sized crystallite, and the amount of heavy C5+ olefin production is reduced by at least 10%. The one or more olefin in the feed is structurally isomerized to at least one skeletal isomer in the reactor.

In other aspects of the presently disclosed method, the method comprises the steps of providing a feed comprising one or more olefin to a reactor containing a zeolite catalyst with a small crystallite, wherein the reactor is maintained at a temperature. The feed is provided at a weight hourly space velocity (WHSV) that is at least three times as fast as the WHSV that is used with conventionally sized zeolite catalysts and the temperature is at least 10° C. lower than the reactor temperature used with conventionally sized catalysts. By changing the catalyst crystalline size, lowering the reactor temperature, and increasing the feed flow, the catalyst cycle is extended by at least 30% compared to processes using catalyst with conventionally sized crystallite, and the amount of heavy C5+ olefin production is reduced by at least 10%. The one or more olefin in the feed is structurally isomerized to at least one skeletal isomer in the reactor.

In other aspects of the presently disclosed method, the method comprises the steps of providing a feed comprising one or more olefin to a reactor containing a zeolite catalyst with a small crystallite, wherein the reactor is maintained at a first temperature that is at least 20° C. lower than a temperature for similar process using a conventionally sized catalyst. The feed is provided at a weight hourly space velocity (WHSV) that is at least 3 times as fast as the WHSV that is used with conventionally sized zeolite catalyst. By changing the zeolite catalyst crystalline size and increasing the feed flow, the catalyst cycle is extended by at least 30% compared to processes using a catalyst with conventionally sized crystallite, and the amount of heavy C5+ olefin production is reduced by at least 10%. The one or more olefin in the feed is structurally isomerized to at least one skeletal isomer in the reactor.

In some aspects of the present methods, the amount of heavy C5+ olefins produced by the isomerization process is decreased by at least 10%, by at least 20%, by at least 30%, or by at least 40%, compared to methods using catalysts with bigger, conventionally sized crystallites.

With the smaller crystallite size and/or faster fed rate and/or lower reactor temperature, the isomerization can be carried out for a longer period of time before decoking of the zeolite catalyst, also known as catalyst regeneration, is needed. In some aspects of the present methods, the length of time that the catalyst can be used before being regenerated, also called the catalyst cycle, is extended by at least 30%, at least 40%, at least 50%, or at least 60%, compared to methods using zeolites with bigger, conventionally sized crystallite. Alternatively, the catalyst cycle is extended by at least 3 days, 4 days, 5 days, 6 days, 7 days, or 8 days, compared to methods using zeolites with bigger, conventionally sized crystallites.

In some aspects of the present method, the catalyst cycle is at least seventeen days (˜2.5 weeks), at least twenty-one days (3 weeks), or at least twenty-five days (˜3.5 weeks), when the WHSV is at least 7 hr⁻¹.

In some aspects of the present methods, the yield of the skeletal isomer product can be 5 to 20% higher than using zeolites with bigger, conventionally sized crystallites.

In some aspects of the present methods, the olefin feed comprises branched, iso-olefins, wherein the skeletal isomerization process converts the branched, iso-olefins to unbranched, linear olefins, which are also referred to as normal olefins. In other aspects of the present methods, the olefin feed comprises linear olefins which are then converted to branched iso-olefins during the novel skeletal isomerization process. The olefins in either feed can have 2 to 10 carbons. The feed may also include other hydrocarbons such as alkanes, other olefins, aromatics, hydrogen, and inert gases.

In other aspects of the present methods, the catalyst used in the isomerization processes can be used alone or combined with a refractory oxide as a binder. The binder that can be used in this disclosure comprises silica, silica-alumina, bentonite, kaolin, bentonite with alumina, montmorillonite, attapulgite, titania and zirconia. The weight ratio of binder material and zeolite can range from 1:10 to 10:1. In embodiments, the weight ratio of binder material and zeolite is 1:5 to 5:1.

The present methods and systems include any of the following embodiments in any combination(s) of one or more thereof:

A skeletal isomerization process comprising the steps of feeding, at a weight hourly space velocity (WHSV) between about 7 to about 30 hr⁻¹, a hydrocarbon feed comprising at least one olefin to a reactor at a known temperature containing an isomerization zeolite catalyst that has a crystallite size that is less than 1 μm in diameter in all directions; and isomerizing the at least one olefin to at least one skeletal isomer product in the reactor for at least one catalyst cycle.

A skeletal isomerization process comprising the steps of feeding, at a weight hourly space velocity (WHSV) between about 7 to about 30 hr⁻¹, a hydrocarbon feed comprising at least one olefin to a reactor at a known temperature and containing an isomerization zeolite catalyst that has a crystallite size that is less than 1 μm in diameter in all directions; and isomerizing the at least one olefin to at least one skeletal isomer product in the reactor for at least one catalyst cycle, wherein the catalyst cycle is at least twenty-one days (three weeks).

A skeletal isomerization process comprising the steps of feeding, at a weight hourly space velocity (WHSV) between about 7 to about 30 hr⁻¹, a hydrocarbon feed comprising at least one olefin to a reactor containing an isomerization zeolite catalyst that has a crystallite size that is less than 1 μm in diameter in all directions; and isomerizing the at least one olefin to at least one skeletal isomer product in the reactor for at least one catalyst cycle, wherein the catalyst cycle is at least seventeen days, wherein the temperature of the reactor is between about 380° C. and 425°.

A skeletal isomerization process comprising the steps of feeding, at a weight hourly space velocity (WHSV), a hydrocarbon feed comprising at least one olefin to a reactor containing an isomerization zeolite catalyst that has a crystallite size that is less than 1 μm in diameter in all directions; and isomerizing the at least one olefin to at least one skeletal isomer product in the reactor for at least one catalyst cycle. The WHSV when using the small crystallite size catalyst is at least three times as an isomerization zeolite catalyst that has a crystallite size that is 1 μm or larger in diameter.

Any of the methods described herein, wherein the WHSV is about 7 to about 14 hr¹.

Any of the methods described herein, wherein the WHSV is about 14 hr⁻¹.

Any of the methods described herein, wherein the catalyst cycle is at least 30% longer as compared to a process using an isomerization zeolite catalyst that has a crystallite size that is 1 μm or larger in diameter.

Any of the methods described herein, wherein the skeletal isomerization process produces heavy compounds having 5 or more carbon atoms (“C5+ heavies”) and the production of C5+ heavies is reduced by at least 5% compared to a skeletal isomerization process using an isomerization zeolite catalyst that has a crystallite size that is 1 μm or larger in diameter.

Any of the methods described herein, wherein the yield of at least one skeletal isomer from the skeletal isomerization process is increased by at least 5% as compared to a process using an isomerization zeolite catalyst that has a crystallite size that is 1 μm or larger in diameter.

Any of the methods described herein, further comprising the step of recovering the skeletal isomer product from the reactor.

Any of the methods described herein, wherein the skeletal isomer product comprises 1-butene and 2-butene.

Any of the methods described herein, wherein the skeletal isomer product comprises isobutylene.

Any of the methods described herein, wherein the at least one olefin is a linear olefin.

Any of the methods described herein, wherein the at least one olefin is 1-butene and 2-butene.

Any of the methods described herein, wherein the at least one olefin is isobutylene.

Any of the methods described herein, wherein the hydrocarbon feed comprises at least 40 wt. % isobutylene.

Any of the methods described herein, wherein the hydrocarbon feed further comprises alkanes, aromatics, hydrogen and other gases.

Any of the methods described herein, wherein in the at least one olefin is isobutylene and the at least one skeletal isomer product is 1-butene and 2-butene.

Any of the methods described herein, wherein in the at least one olefin comprises 1-butene and 2-butene, and the at least one skeletal isomer product is isobutylene.

Any of the methods described herein, wherein the isomerization zeolite catalyst has a crystallite size that is less than 0.2 μm in diameter in all directions.

Any of the methods described herein, wherein the temperature of the reactor is between about 340° C. to 500° C.

Any of the methods described herein, wherein the temperature of the reactor is between about 380° C. to 425° C.

Any of the methods described herein, wherein the isomerization zeolite catalyst has a silica to alumina ratio from 10:1 to 60:1.

Any of the methods described herein, wherein the isomerization zeolite catalyst is hydrogen form of ferrierite (H-FER).

Any of the methods described herein, wherein the isomerization zeolite catalyst additionally comprises a binder material selected from the group consisting of: silica, silica-alumina, bentonite, kaolin, bentonite with alumina, montmorillonite, attapulgite, titania and zirconia.

Any of the methods described herein, wherein the catalyst cycle is at least seventeen days, at least 21 days, or at least 25 days in length.

This summary is provided to introduce a selection of concepts that are further described below in the detailed description. This summary is not intended to identify key or essential features of the claimed subject matter, nor is it intended to be used as an aid in limiting the scope of the claimed subject matter.

### Definitions

As used herein, the terms “skeletal isomerization” are used interchangeably to refer to an isomerization process that involves the movement of a carbon atom to a new location on the skeleton of the molecule, e.g., from a branched isobutylene skeleton to a linear or straight chain (not branched) butene skeleton. The product in the skeletal isomerization process is a skeletal isomer of the reactant. The term “skeletal isomer” refers to molecules that have the same number of atoms of each element and the same functional groups but differ from each other in the connectivity of the carbon skeleton.

As used herein, “zeolite” means includes a wide variety of both natural and synthetic positive ion-containing crystalline aluminosilicate materials, including molecular sieves. Zeolites are characterized as crystalline aluminosilicates which comprise networks of SiO₄ and A104 tetrahedra in which silicon and aluminum atoms are cross-linked in a three-dimensional framework by sharing of oxygen atoms. This framework structure contains channels or interconnected voids that are occupied by cations, such as sodium, potassium, ammonium, hydrogen, magnesium, calcium, and water molecules. The water may be removed reversibly, such as by heating, which leaves a crystalline host structure available for catalytic activity. The term “zeolite” in this specification is not limited to crystalline aluminosilicates. The term as used herein also includes silicoaluminophosphates (SAPO), metal integrated aluminophosphates (MeAPO and ELAPO), and metal integrated silicoaluminophosphates (MeAPSO and ELAPSO). The MeAPO, MeAPSO, ELAPO, and ELAPSO families have additional elements included in their framework. For example, Me represents the elements Co, Fe, Mg, Mn, or Zn, and El represents the elements Li, Be, Ga, Ge, As, or Ti. An alternative definition would be “zeolitic type molecular sieve” to encompass the materials useful for this disclosure.

As used herein, “H-FER” or “hydrogen form of ferrierite” refers to a hydrogen exchanged ferrierite zeolite.

As used herein, “crystal size” refers to the diameter of the zeolite crystals which exist in a zeolite catalyst; “channel size” refers to the size of the channels in the zeolite structure; and “pore size” refers to the size of the pore, or opening, in the zeolite structure.

As used herein, “coke” refers to the formation of carbonaceous materials on a catalyst surface, particularly inside and around the mouths of the zeolite cages or channels, that leads to the deactivation of the catalyst. As understood in the field, coke is the end product of carbon disproportionation, condensation and hydrogen abstraction reactions of adsorbed carbon-containing material.

As used herein, the terms “decoking” and “catalyst regeneration” refers to the removal of coke from a catalyst's surface. While there are many ways for removing coke from a catalyst, one such method includes reactions of atomic oxygen with “coke” and yields gases such as CO, CO₂ as well as other gaseous products that could be removed.

As used herein, the terms “life cycle of the catalyst”, “catalyst cycle” or “catalyst lifetime” are used interchangeably to refer to the length of time the catalyst is in use before being regenerated.

As used herein, “olefin” refers to any alkene compound that is made up of hydrogen and carbon that contains one or more pairs of carbon atoms linked by a double bond. A “C” followed by a number, in reference to an olefin, refers to how many carbon atoms the olefin contains. For example, a C4 olefin can refer to butene, butadiene, or isobutene. A plus sign (+) is used herein to denote a composition of hydrocarbons with the specified number of carbon atoms plus all heavier components. As an example, a C4+ stream comprises hydrocarbons with 4 carbon atoms plus hydrocarbons having 5 or more carbon atoms.

As used herein, WHSV or “weight hour space velocity” refers to the weight of feed flowing per hour per unit weight of the catalyst. For example, for every 1 gram of catalyst, if the weight of feed flowing is 100 gram per hour, then the WHSV is 100 hr⁻¹.

As used herein, “atmosphere” in the context of pressure refers to 101,325 Pascal, or 760 mmHg, or 14.696 psi.

The terms “heavy olefins” is used to denote compositions of C5+ hydrocarbons, including mono-olefins and diolefins.

The term “conversion” is used to denote the percentage of a component fed which disappears across a reactor.

The term “2-butene” as used herein refers to both cis-2-butene and trans-2-butene.

The term “linear C4 olefin” as used herein refers 1-butene, cis-2-butene and/or trans-2-butene.

The term “normal butene yield” refers to the amount of normal, linear butenes, including 1- and 2-butene, formed during the isomerization process.

As used herein, the term “raffinate” refers to a residual stream of olefins obtained after the desired chemicals/material have been removed. In the cracking/crude oil refining process, a butene or “C4” raffinate stream refers to the mixed 4-carbon olefin stream recovered from the cracker/fluid catalytic cracking unit. The term “Raffinate 1” refers to a C4 residual olefin stream obtained after separation of butadiene (BD) from the initial C4 raffinate stream. “Raffinate 2” refers to the C4 residual olefin stream obtained after separation of both BD and isobutylene from the initial C4 raffinate stream. “Raffinate 3” refers to the C4 residual olefin stream obtained after separation of BD, isobutylene, and 1-butene from the initial C4 raffinate stream. In some embodiments of the present disclosure, the isobutylene separated from Raffinate 1 can be used as a source for the skeletal isomerization process, especially when C4 alkanes have first been removed.

As used herein, “binder” refers to the material used in the catalyst and provide necessary mechanical strength and/or resistance towards attrition loss. Common binders include clays, kaolin, attapulgite, boehmite, aluminas, silicas or combinations thereof. Binders are added in quantities higher than 20% in weight to reach the mechanical strength needed and form a homogeneous and plastic mixture. Binders used herein include, but are not limited to, silica, silica-alumina, bentonite, kaolin, bentonite with alumina, montmorillonite, attapulgite, titania, zirconia, and combinations thereof.

As used herein, “silica” refers to SiO₂, “alumina” refers to Al₂O₃, “attapulgite” refers to a magnesium aluminum phyllosilicate, “titania” refers to titanium dioxide, and “zirconia” refers to zirconium dioxide.

Use of the term “optionally” with respect to any element of a claim means that the element is required, or alternatively, not required, both alternatives being within the scope of the claim.

Numbers and ranges disclosed above may vary by some amount. Whenever a numerical range with a lower limit and an upper limit is disclosed, any number and any included range falling within the range is specifically disclosed. In particular, each range of values (of the form, “from about a to about b,” or, equivalently, “from approximately a to b,” or, equivalently, “from approximately a-b”) disclosed herein is to be understood to set forth each number and range encompassed within the broader range of values.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The terms in the claims have their plain, ordinary meaning unless otherwise explicitly and unambiguously defined by the patentee. Moreover, the indefinite articles “a” or “an”, as used in the claims, are defined herein to mean one or more than one of the elements that it introduces. If there is any conflict in the usages of a word or term in this specification and one or more patent or other documents, the definitions that are consistent with this specification should be adopted.

The following abbreviations are used herein:

| ABBREVIATION | TERM |
|---|---|
| B1 | 1-butene |
| B2 | 2-butene |
| EFF | effluent |
| FD | feed |
| H-FER | Hydrogen ferrierite |
| IB1 | Isobutylene |
| PO/TBA | propylene oxide/t-butyl alcohol |
| WHSV | Weight hour space velocity (mass feed rate per hour |
| | per mass of catalyst |
| wt. % | weight percent |

### BRIEF DESCRIPTION OF THE DRAWINGS

FIG. 1A. The conversion rate of isobutylene to normal butene of one embodiment of the present disclosure.

FIG. 1B. Yield of isobutylene of one embodiment of the present disclosure.

FIG. 1C. Yield of C5+ heavies of one embodiment of the present disclosure.

FIG. 2A. Conversion rate of isobutylene to normal butene between embodiments of the present disclosure utilizing different space velocities.

FIG. 2B. Comparison of yield of isobutylene for embodiments of the present disclosure utilizing different space velocities.

FIG. 2C. Comparison of yield C5+ heavies for embodiments of the present disclosure utilizing different space velocities.

### DETAILED DESCRIPTION

The disclosure provides a skeletal isomerization method for isomerizing olefins using a zeolite catalyst with a small crystallite size, and a faster feed flow and/or lower reactor temperature, to increase the lifetime of the catalyst before regeneration is needed. In some embodiments of the presently disclose method, a reduction in the formation of the heavy C5+ olefins occur while increasing the formation of the skeletal isomer products. In some embodiments of the presently disclose method, the smaller zeolite catalyst is more active than conventional sized catalysts, resulting in less catalyst material being needed for the same feed flow rate.

Conventional skeletal isomerization processes, both forward isomerization of linear olefins to branch olefins and reverse isomerization of branched olefins to linear olefins, employ catalysts, such as zeolites, with large crystallites that have a diameter of 1 μm or greater. These zeolite catalysts can be used with or without a refractory oxide binder material such as silica or alumina, and many are commercially available. However, these zeolite catalysts are susceptible to quick coking and subsequent blocking of pores, which lead to low cycle times before the catalyst must be de-coked and regenerated. Further, processes using zeolite catalysts with conventional crystallite sizes can also result in an undesired by-product formation of heavy C5+ olefins, particularly in the beginning of the cycle for reverse isomerization.

The presently disclosed methods overcome the issues in the conventional isomerization process by using a zeolite catalyst with a “small” crystallite size that is defined as being less 1 μm in diameter in all directions. This is a smaller crystallite size than what is conventionally used, and is more active. This results in the need for less catalyst material that a conventionally sized catalyst for the same feed rate. Further, increase in activity also results in a longer catalyst cycle. However, in some methods, it may not improve the reaction product yield or selectively of reaction product formation. Thus, the presently disclosed method further includes using a faster hydrocarbon feed flow through the reactor than the conventional isomerization methods and/or decreasing the reactor temperature compared to conventional isomerization methods. These changes to the zeolite catalyst and the process conditions not only increase the yield of reaction productions while reducing the formation of C5+ olefins, but also increase the catalyst cycle compared to methods using a catalyst with conventional crystallite sizes. In some embodiments, the increase in the length of the catalyst cycle and the use of less catalyst material is retained even with the changes to the feed or reactor temperatures.

Without being bound by theory, it is thought that the smaller zeolite crystallite size catalyst is utilized in a different way than the conventional larger crystallite zeolites. Contrary to conventional belief that catalyst of smaller crystallite sizes might have diffusion limitation due to their sizes, the results shown in this disclosure indicate the opposite is true. It is proposed that the smaller size provides less surface area for an unselective transformation to coke, therefore possibly increasing the life of the catalyst. It is also proposed that the smaller crystallites provide an increase in active site density affording a higher activity. Additionally, it is proposed that a preferential coking could occur at specific locations in the zeolite catalyst, such that once the preferential coking occurs, further coking is reduced.

The rate of isomerization is also increased with the use of the smaller zeolite crystallite size of this disclosure. In some embodiments, the rate of isomerization is increased by 5 to 20% as compared to conventionally sized zeolite catalysts. In some embodiments, the rate of isomerization is increased by at least 10% as compared to conventionally sized zeolite catalysts.

The life cycle of the catalyst, also called the catalyst cycle, of this disclosure can also be increased as compared to conventionally sized catalyst in the isomerization process. In some embodiments, the life cycle of the catalyst is at least 50% longer than a conventionally sized catalyst. In some embodiments, the life cycle of the catalyst is at least 75% longer than a conventionally sized catalyst. In some embodiments, the life cycle of the catalyst is at least 100% longer than a conventionally sized catalyst.

Alternatively, the life cycle of the catalyst is extended by at least 3 days, 4 days, 5 days, 6 days, 7 days, or 8 days, compared to methods using zeolites with bigger, conventionally sized crystallites. In some aspects of the present method, the catalyst cycle is at least seventeen days (˜2.5 weeks), at least twenty-one days (3 weeks), or at least twenty-five days (˜3.5 weeks), when the WHSV is at least 7 hr⁻¹.

The yield of linear olefins by using the catalyst of this disclosure is increased due to the longer life cycle and higher reaction rate. In some embodiments, the yield of linear olefin by using the catalyst of this disclosure can be 5 to 20% higher than using a conventionally sized catalyst. In some embodiment, the yield of linear olefin using the catalyst of this disclosure is at least 10% higher than using a conventionally sized catalyst.

The amount of catalyst material when using the small crystallite sized catalyst of this disclosure is reduced compared to a conventional sized catalyst, for the same feed flow. In some embodiments, the amount of the catalyst of this disclosure needed for a given feed flow can be 5 to 67% less than using a conventionally sized catalyst. In other embodiment, the amount of the catalyst of this disclosure needed for a given feed flow is at least 33% less than using a conventionally sized catalyst. In some embodiments, the skeletal isomerization process uses about one-third to about two-thirds less of the small crystallite size zeolite catalyst than the amount of conventionally sized catalyst, for the same process conditions.

In some embodiments, the novel method presently disclosed comprises the steps of feeding a hydrocarbon feed that has at least one olefin into to a reactor having an isomerization zeolite catalyst with a small crystallite size that is less than 1 μm in diameter in all directions at a hydrocarbon weight hour space velocity (WHSV) in the range of from 1 to 30 hr⁻¹, wherein the reactor is maintained at a first temperature and a first pressure, and collecting one or more skeletal isomer olefin product. The at least one olefin in the feed can have two to ten carbons, and, during the feeding steps, a portion of the at least one olefin is isomerized into the at least one skeletal isomer olefin product. For example, if the at least one olefin is an iso-olefin such as isobutylene, then the skeletal isomer olefin product will be a linear olefin such as 1- or 2-butene. If the at least one olefin is a linear olefin such as 2-butene, then the skeletal isomer olefin product will be an iso-olefin such as isobutylene.

In some embodiments, the novel method presently disclosed comprises the steps of feeding a hydrocarbon feed that has at least one olefin into to a reactor having an isomerization zeolite catalyst with a small crystallite size that is <0.2 μm in diameter in all directions at a first hydrocarbon weight hour space velocity, wherein the reactor is maintained at a first temperature and a first pressure, and collecting one or more skeletal isomer olefin product. The at least one olefin in the feed can have two to ten carbons, and, during the feeding steps, a portion of the at least one olefin is isomerized into the at least one skeletal isomer olefin product.

In some embodiments, the novel method presently disclosed comprises the steps of feeding a hydrocarbon feed that has at least one olefin into to a reactor having an isomerization zeolite catalyst with a small crystallite size that is less than 1 μm in diameter in all directions at a hydrocarbon weight hour space velocity (WHSV) in the range of from 1 to 30 hr⁻¹, wherein the reactor is maintained at a temperature between 340° C. and 500° C. and a pressure between zero to about 1034 kPa (150 psig), and collecting one or more skeletal isomer olefin product. The at least one olefin in the feed can have two to ten carbons, and, during the feeding steps, a portion of the at least one olefin is isomerized into the at least one skeletal isomer olefin product.

In some embodiments, the novel method presently disclosed comprises the steps of feeding a hydrocarbon feed that has at least one olefin into to a reactor having an isomerization zeolite catalyst with a small crystallite size that is less than 1 μm in diameter in all directions at a first hydrocarbon weight hour space velocity, wherein the reactor is maintained at a first temperature and a first pressure, and collecting one or more skeletal isomer olefin product, wherein the catalyst cycle is at least 50% longer than a method that does not use the small crystallite size. The at least one olefin in the feed can have two to ten carbons, and, during the feeding steps, a portion of the at least one olefin is isomerized into the at least one skeletal isomer olefin product.

In some embodiments, the novel method presently disclosed comprises the steps of feeding a hydrocarbon feed that has at least one olefin into to a reactor having an isomerization zeolite catalyst with a small crystallite size that is less than 1 μm in diameter in all directions at a first hydrocarbon weight hour space velocity, wherein the reactor is maintained at a first temperature and a first pressure, and collecting one or more skeletal isomer olefin product, wherein the catalyst cycle is at least 50% longer than a method that does not use the small crystallite size and the first hydrocarbon weight hour space velocity is at least 3 times as fast as a method that does not use the small crystallite size. The at least one olefin in the feed can have two to ten carbons, and, during the feeding steps, a portion of the at least one olefin is isomerized into the at least one skeletal isomer olefin product.

More details on the skeletal isomerization process conditions and feeds are provided below.

Hydrocarbon Feedstream:

The presently described methods are for the skeletal isomerization (both forward and reverse) of olefins, also known as alkenes. Thus, the hydrocarbon feedstream, or feed, used herein may comprises at least one olefin that will be isomerized into a skeletal isomer thereof. For example, an iso-olefin is a skeletal isomer of a linear olefin, and vice versa. In some embodiments, the at least one olefin in the hydrocarbon feed has two to ten carbon atoms.

In some embodiments, the hydrocarbon feed comprises unbranched linear, or normal, olefins having two to ten carbons, as well as other hydrocarbons such as alkanes, di-olefins, aromatics, hydrogen, and inert gases. In other embodiments, the feed comprises at least 40 wt. % of linear C4 olefins, as well as other hydrocarbons such as alkanes, other olefins, aromatics, hydrogen, and inert gases. Alternatively, the feed comprises at least 55 wt. % of linear C4 olefins, at least 70 wt. % of linear C4 olefins, at least 85 wt. % of linear C4 olefins, at least 95 wt. % of linear C4 olefins, or at least 99 wt. % of linear C4 olefins.

In other embodiments, the hydrocarbon feed used herein comprises branched olefins, also known as “iso-olefins”. In this disclosure, the branched olefins can have four to ten carbon atoms. In some embodiments, the feed used herein comprises a methyl-branched iso-olefin. In some embodiments of the disclosure, the feed contains isobutylene. As before, the hydrocarbon feed used in some embodiments of the disclosure may also include other hydrocarbons such as alkanes, di-olefins, and aromatics, as well as hydrogen and other gases.

In some embodiments of the disclosure, the feed comprises at least 40 wt. % isobutylene, at least 55 wt. % isobutylene, at least 70 wt. % isobutylene, at least 85 wt. % isobutylene, at least 95 wt. % isobutylene, or at least 99 wt. % isobutylene. The isobutylene can be from any source. In some embodiments, the isobutylene comes from a Raffinate 1 stream derived from a cracker/fluid catalytic cracking unit and has had its C4 alkanes removed. Alternatively, the isobutylene can come from a stream derived from a propylene oxide/t-butyl alcohol (PO/TBA) plant. The dehydration of the t-butyl alcohol can result in a more purified isobutylene stream than a stream sourced from a cracker.

Isomerization Catalyst:

Conventional skeletal isomerization zeolite catalysts have large crystallite sizes that are 1 μm or greater (≥1 μm) in all directions. However, the isomerization catalysts used in the presently disclosed process differ from conventional isomerization catalysts in that the present process utilizes an isomerization catalyst with a smaller crystallite size (less than 1 μm in diameter in all directions) than the conventional catalyst.

The crystallite size for the catalyst used in the presently disclosed methods has a diameter less than 1 μm, less than 0.5 μm, less than 0.3 μm, or less than 0.2 micron. In addition to the smaller crystallite, the catalyst used in the presently disclosed methods may also have a silica to alumina ratio (SAR) of about 10:1 to about 60:1. In some embodiments, the SAR of the catalyst used in the presently described methods is about 10, about 20, about 40 or about 50. In other embodiments, the SAR is limited to 10 to 50 due to the small crystallite size of the catalyst.

In some embodiments of the presently disclosed process, the catalyst has a crystallite size that is about 0.2 μm in diameter and a SAR of about 20. Alternatively, the catalyst has a crystallite size that is about 0.2 μm in diameter, a SAR of about 20, a surface area ranging from about 300 m²/g to about 450 m²/g and a micropore volume ranging from about 0.10 cc/g to about 0.20 cc/g. In some embodiments of the present disclosure, the H-FER catalyst has a Na₂O content in the range of 0 to 0.10 wt. %. In some embodiments of the present disclosure, the H-FER catalyst has a Na₂O content in the range of 0 to 0.05 wt. %. In some embodiments of the present disclosure, the H-FER catalyst has a Na₂O content in the range of 0.05 to 0.10 wt. %. In some embodiments of the present disclosure, the H-FER catalyst has a Na₂O content of 0 wt. %. In some embodiments of the present disclosure, the H-FER catalyst has a Na₂O content less than 0.04 wt. %, a SAR of about 25, an XRD crystallinity of 96%, a BET surface area of 421 m²/g, a crystal size (SEM) less than 200 nm, and a loss on ignition of about 9 wt. %. All relative amounts defined within this paragraph are based upon the total weight of the H-FER catalyst.

The small crystallite sized isomerization catalyst used in embodiments of this disclosure includes catalysts suitable to skeletally isomerize olefins. This includes isomerizing iso-olefins to linear, or normal, olefins (unbranched) and vice versa.

In some embodiments of the present disclosure, the isomerization catalyst is a smaller version of FER called “small ferrierite” or s-FER. The s-FER has the same crystal structure as the conventionally sized ferrierite but with a crystallite size that is less than 1 μm. The s-FER can also be in the hydrogen form. Conversion of ferrierite to its hydrogen form, H-FER, replaces sodium cations with hydrogen ions in the crystal structure, making it more acidic.

In some embodiments, the isomerization catalyst is a H-FER with a small crystallite size of about 0.2 μm or less in diameter and a silica to alumina ratio of about 10 to about 60. Alternatively, the isomerization catalyst is a H-FER with a small crystallite size of about 0.2 μm or less in diameter and a silica to alumina ratio of about 20.

Various ferrierite zeolites (“FER”), including the hydrogen form of ferrierite, are described in U.S. Pat. Nos. 3,933,974, 4,000,248, and 4,942,027 and patents cited therein. Various methods are provided which teach procedures for preparing H-ferrierite, including U.S. Pat. Nos. 4,251,499, 4,795,623 and 4,942,027, incorporated herein by reference in their entirety. In some embodiments of the present disclosure, the zeolite catalyst may be a H-FER catalyst prepared in accordance with U.S. Pat. No. 9,827,560 B2, incorporated herein by reference in its entirety. In other embodiments of the present disclosure, the zeolite catalyst is a commercially available catalyst including, but not limited to, ZD18018TL from Zeolyst International.

The small crystallite size zeolite catalyst used in embodiments of the present disclosure may be used alone or suitable combined with a refractory oxide that serves as a binder material. Suitable refractory oxides include, but are not limited to, natural clays, such as bentonite, montmorillonite, attapulgite, and kaolin; alumina; silica; silica-alumina; hydrated alumina; titania; zirconia and mixtures thereof. The weight ratio of binder material and zeolite suitably ranges from 1:10 to 10:1. In some embodiments of the disclosure, the weight ratio of binder to zeolite is in the range of 1:10 to 5:1, the range of 3:5 to 10:1, or the range of 3:5 to 8:5. In some embodiments of the present disclosure, the binder comprises from 10 wt. % to 20 wt. % of the catalyst-binder combination. In some embodiments of the present disclosure, the binder comprises from 10 wt. % to 15 wt. % of the catalyst-binder combination. In some embodiments of the present disclosure, the binder comprises from 15 wt. % to 20 wt. % of the catalyst-binder combination. In some embodiments of the present disclosure, the binder comprises from 13 wt. % to 17 wt. % of the catalyst-binder combination.

Despite the difference in the crystallite size, the isomerization catalyst in the presently disclosed methods, when combined with at least one binder, can be any shape used with conventional isomerization catalysts. This includes, but is not limited to, spheres, pellets, tablets, platelets, cylinders, helical lobed extrudate, trilobes, quadralobes, multilobed (5 or more lobes), and combinations thereof. In some embodiments, the isomerization catalyst is a trilobed, quadralobe, or multilobed extrudate.

Operating Conditions for Skeletal Isomerization Process:

In some embodiments of the disclosure, the hydrocarbon feed may be contacted with the isomerization catalyst under reaction conditions effective to skeletally isomerize the olefins therein. This contacting step may be conducted in the vapor phase by bringing a vaporized feed into contact with the solid isomerization catalyst. The hydrocarbon feed and/or catalyst can be preheated as desired.

The isomerization process of the disclosure may be carried out in a variety of reactor types. In some embodiments of the disclosure, the reactor is a packed bed reactor. In some embodiments of the disclosure, the reactor is a fixed bed reactor. In some embodiments of the disclosure, the reactor is a fluidized bed reactor. In some embodiments of the disclosure, the reactor is a moving bed reactor. In embodiments of the disclosure using a moving bed reactor, the catalyst bed may move upwards or downwards.

The temperature of the reactor can vary from about 250° C. to about 600° C., or from about 380° C. to about 425° C. Alternatively, the reactor temperature for the isomerization is between about 250° C. to about 420° C., about 400 and 600° C., or about 340° and 500° C. In yet another alternative, the reactor temperature is about 418° C.

In other embodiments, the temperature of the reactor is at least 20° C. less than the temperature used in conventional isomerization processes. In other embodiments, the temperature of the reactor is at least 40° C. less than the temperature used in conventional isomerization processes. Alternatively, the temperature of the reactor is at least 25° C., at least 35° C., at least 45° C., or at least 55° C. less than the reactor temperature used in conventional isomerization processes.

The reaction pressure conditions can vary from about zero to about 1034 kPa (150 psig), or from about zero to about 345 kPa (50 psig). Alternatively, the reaction pressure for the isomerization is between about 34 kPa (5 psig) to about 345 kPa (50 psig), about 34 kPa (5 psig) to about 83 kPa (12 psig), 55 kPa (8 psig) to about 138 kPa (20 psig), or 55 kPa (8 psig) to about 97 kPa (14 psig). In yet another alternative, the pressure is about 69 kPa (10 psig).

In some embodiments of the present disclosure, the smaller crystallite catalyst can be combined with a faster weight hourly space velocity (WHSV) of the hydrocarbon feed rate to improve the yield while prolonging the life of catalyst. The weight hourly space velocity feed rates of the olefin feed can range from about 1 to about 200 hr⁻¹, with or without a conventional diluent. In some embodiments, the weight hourly space velocity feed rates are from about 1 to about 30 hr⁻¹. In some embodiments, the weight hourly space velocity feed rates are from about 7 to about 14 hr⁻¹, or about 14 hr⁻¹.

In other embodiments, the weight hourly space velocity feed rates are at least 3 times the feed rates used in conventional isomerization processes. In other embodiments, the weight hourly space velocity feed rates are at least 3 to 8 times the feed rates used in conventional isomerization processes. Alternatively, the weight hourly space velocity feed rates are at least 3.5 times, at least 4 times, at least 7 times, or at least 8 times the feed rates used in conventional isomerization processes.

By performing a skeletal isomerization using the steps above and a catalyst with a small crystallite size, the catalyst cycle and yield of the skeletal isomer product increases compared to an isomerization process that uses catalysts with conventionally sized crystallites. The catalyst cycle can be increased by at least 50%, 75%, or 100%, compared to an isomerization process that uses catalysts with conventionally sized crystallites. The yield of skeletal isomer product olefins obtained using embodiments of the disclosure may be at least 5 to 20% greater compared to an isomerization process with a conventional catalyst. In some embodiments of the disclosure, the yield of skeletal isomer product olefins obtained may be at least 10% greater than a similar isomerization process that does not include a catalyst with the small crystallite size described in this disclosure.

In some embodiments of the present disclosure, the use of the smaller crystallite catalyst can increase the life of catalyst to at least seventeen days (˜2.5 weeks), at least twenty-one days (3 weeks), or at least twenty-five days (˜3.5 weeks), when the WHSV is at least 7 hr⁻¹.

Using the above described methods, the skeletal isomerization process is improved because the catalyst cycle is longer, allowing for a greater amount of structurally isomerized product, also called skeletal isomer olefin product, to be formed. In some embodiments, when the feed comprises C4 olefins, a greater amount of the desired structurally isomerized product can be formed while forming less heavy C5+ olefins. This leads to a more cost-effective isomerization process for generating greater amounts of structurally isomerized C4 olefins.

### EXAMPLES

The following examples are included to demonstrate embodiments of the appended claims using the above described system and methods of increasing the yield of isomerization products for an isobutylene feed and the catalyst cycle. The example is intended to be illustrative, and not to unduly limit the scope of the appended claims. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

Hydrocarbon Feed.

For each of the Examples below, the feed comprised 99.95 wt. % of isobutylene. The skeletal isomer product olefins for such a feed composition include 1-butene and 2-butene (including both trans-2-butene and cis-2-butene).

Calculations.

For each of the Examples below, the conversion of reactants to products is calculated. Without being bound by theory, it is believed that during the isomerization reaction, equilibrium is achieved between, for example, the isobutylene, 1-butene and trans- and cis-2-butene. Therefore, the calculation of conversion reflects the feed (FD) and effluent (EFF) concentrations of 1-butene (B1), 2-butene (B2), and isobutylene (IB1). Conversion is calculated as:

$$\% isobutylene Conversion = \frac{( wt \% IB 1 ) FD - ( wt \% IB 1 ) EFF}{( wt \% IB 1 ) FD} \times 100$$

Yield is calculated as

$$\% linear butene Yield = \frac{( wt \% B 1 + wt \% B 2 ) EFF - ( wt \% B 1 + wt \% B 2 ) FD}{( wt \% IB 1 ) FD} \times 100$$

Development of equivalent equations for other olefin reactants and skeletal isomer products are well within the abilities of one with skill in the art.

For the catalyst cycle determination, a 30% conversion rate was used as a practical economic cut-off before decoking is required. This percentage was based on equipment that was used in the present examples. However, other cutoffs or means for measuring when a catalyst needs to be regenerated are possible, depending on the equipment being used and the amount of hydrocarbon feed that is being recycled.

### Example 1: Catalyst with Small Crystallite Size

A series of skeletal isomerization reactions were performed using isomerization catalysts with different crystallite sizes. The operational conditions for both reactions were exactly the same, with the only difference being the crystallite size.

Comparative Example 1 used a commercially available H-FER catalyst with a conventional crystallite size that is greater the 1 μm. In contrast, Example 1 used a H-FER catalyst with a small crystallite size that was about 0.2 μm. Both catalysts had a trilobed extrudate shape. The H-FER catalyst in Example 1 had silica:alumina ratio of 20. The H-FER catalyst in Comparative Example 1 had silica:alumina ratio of 90. No catalyst pretreatment was performed for either reaction.

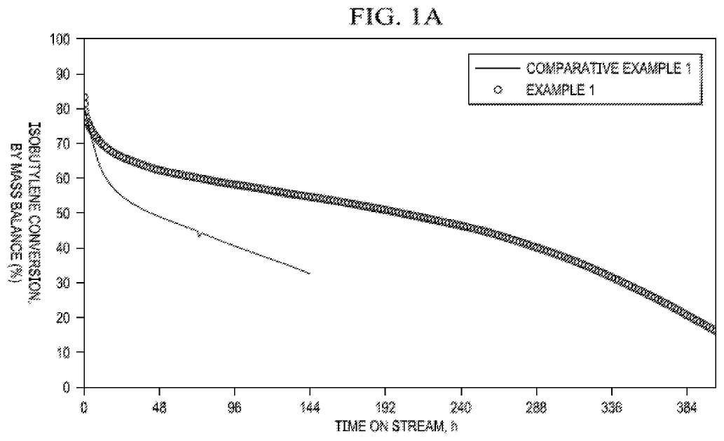

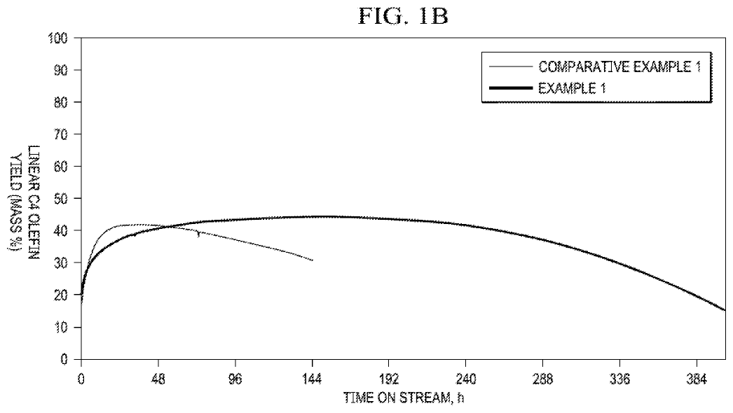

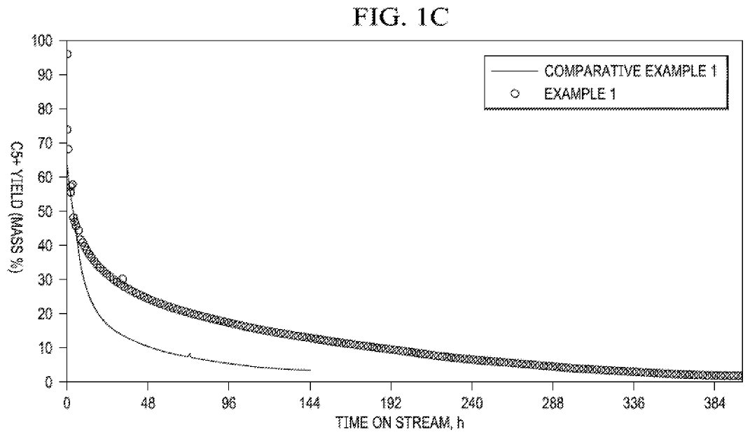

For both reactions, the isobutylene feed was fed through a fixed bed reactor held at a temperature of approximately 418° C. The isobutylene feed was maintained at a WHSV of 7 hr⁻¹ (7 g isobutylene/g catalyst/hr) for both reactions. The results for both reactions are displayed in FIGS. 1A-C.

The conversion rate of isobutylene to linear butenes and the catalyst cycle for each catalyst is displayed in FIG. 1A. The isobutylene conversion for Example 1 is about 10% higher during its catalyst cycle than that in Comparative Example 1. However, the catalyst cycle is much longer. Using 30% conversion rate as a practical economic cut-off for minimum of acceptable catalyst before decoking is required, the catalyst of Example 1 lasted about 336 hours, whereas the conventional catalyst of Comparative Example 1 lasted about 144 hours. The difference is 192 hours, or 8 days. In other words, the life cycle of the catalyst with the small crystallite size is able to more than double the life of a conventional catalyst ((336-144)/144*100%=133%). The doubling of life cycle translates into cost saving in both the amount of catalyst and the fewer interruption on operation.

The yield of reaction products is shown in FIGS. 1B and 1C. The yield of linear butenes in the reaction for Example 1 is much higher than that in the Comparative Example 1, as shown in FIG. 1B. The conventional catalyst in Comparative Example 1 reaches the highest yield of linear butenes sooner than the small crystallite catalyst of this disclosure, but the yield for Comparative Catalyst 1 quickly drops thereafter. In contrast, the yield of linear butenes for Example 1 slowly increases, before reaching a maximum well after the catalyst cycle for Comparative Example 1 ends. Thus, the yield of linear butenes for Example 1 is much larger than that of Comparative Example 1.

As seen in FIG. 1C, the production of the undesired heavy C5+ olefins also increased for Example 1. Heavy C5+ olefins are byproducts that have to be separated by other processes downstream for use in low value gasoline or other products. The amount of C5+ olefins producing using the small crystallite catalyst is about 10% higher than the conventionally sized catalyst. It is believed that this increase is due to the lower SAR ratio. The small crystallite size limits the SARs, thus other modifications to the process would be needed to address the increase in heavy C5+ olefins. One potential modification is described below in Example 2.

### Example 2: Faster WHSV

The results in Example 1 show that decreasing the crystallite size of the catalyst will increase the catalyst cycle, as compared to a similar process using a catalyst with a conventional crystallite size, and subsequently increase the yield of linear butenes. However, the smaller crystallite size also increased the production of the undesirable heavy C5+ olefins. As such, this example is directed to modifying the isomerization conditions to reduce the production of heavy C5+ olefins without sacrificing the benefits of a catalyst with a smaller crystallite size.

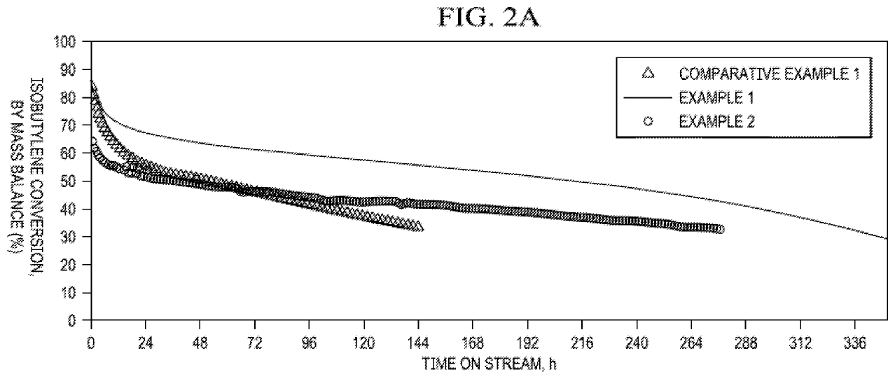

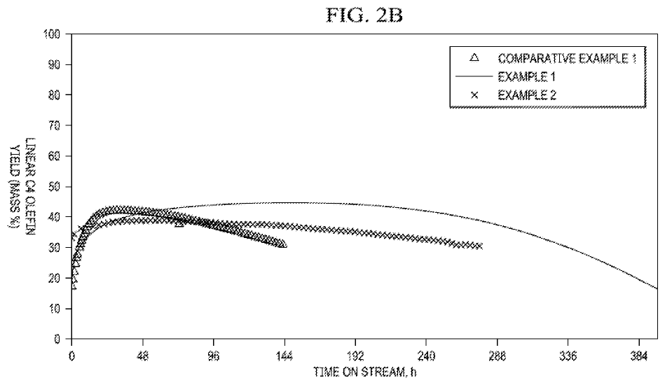

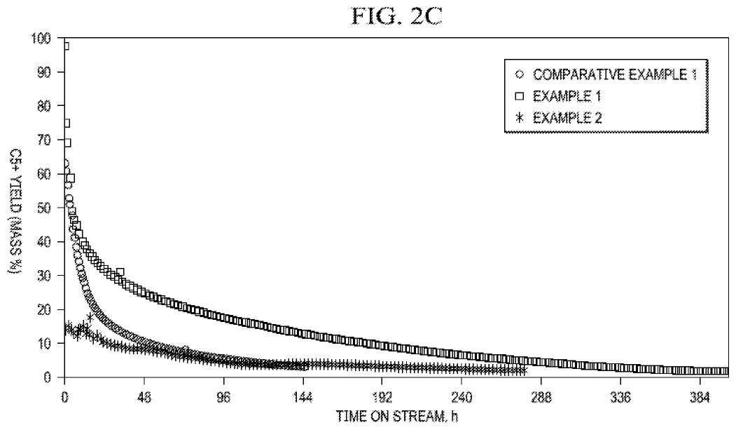

Example 2 is an isomerization reaction of isobutylene that was ran under the same conditions and with the same isomerization catalyst as Example 1, except the WHSV was set at 14 hr⁻1 (14 g isobutylene/g catalyst/hr). The reactor temperature was maintained at approximately 406-418° C. The results are shown in FIGS. 2A-C.

FIG. 2A displays the isobutylene conversion and catalyst cycle for Examples 1 and 2, as well as Comparative Example 1. FIG. 2B displays the yield of linear butene for these examples, and FIG. 2C displays the yield of heavy C5+ olefins for the same examples.

As can be seen in FIG. 2A, the conversion rate of isobutylene and the catalyst cycle in Example 2, when the WHSV is 14 hr⁻¹ and is twice as fast as the other examples, is between that of Comparative Example 1 and Example 1. While the catalyst cycle in Example 2 is less than that of Example 1, it is still 83% longer than that of Comparative Example 1 which is a catalyst with a conventional crystallite size ((264-144)/144*100%=83%). Thus, the longer life cycle benefit of the catalyst with a smaller crystallite size was retained even though the feed rate was doubled.

FIG. 2B shows the yield of normal butene and C5+ heavies with respect to WHSV=7 and WHSV=14. Similar to Example 1, Example 2 had a lower yield than Comparative Example 1 during the early part of the isomerization reaction. However, the longer catalyst cycle allowed for Example 2 to ultimately have a higher yield than Comparative Example 1. Thus, the higher linear butene yield of the catalyst with a smaller crystallite size was retained.

FIG. 2C displays the yield of heavy C5+ olefins. Unlike Example 1, Example 2 had a much lower yield of C5+ olefins. The amount of C5+ olefins was significantly lower during the beginning of the reaction. Example 1 had an initial value of about 60%, whereas Example 2 was four times lower, with a value of 15%. This lower production of heavy C5+ olefins continued through the catalyst cycles. At the 240-hour mark, Example 2 produced about 191 grams of heavy C5+ olefins. In contrast, Example 1, at the same time in the reaction, produced about 298 grams. By doubling the WHSV, the amount of heavy C5+ olefins was reduced by about 35%. Regarding Comparative Example 1, Example 2 has a lower starting yield, however it should overtake the yield in Comparative Example 1 because of the longer cycle length.

Thus, the combination of an isomerization catalyst with a small crystallite size and a faster WHSV resulted in an increase in the catalyst cycle length, an increase in the yield of linear butenes, and a decrease in heavy C5+ olefins.

### Prophetic Examples

Experiments can be run under the same conditions as Example 2, except for lowering the reactor temperature to further increase the catalyst cycle while maintaining the higher skeletal isomer product yield and lower C5+ heavies yield.

Additional experiments can be run using the same conditions as either Example 1 or 2, but with a catalyst with the small crystallite size and a higher SAR. The higher SAR may further lower the C5+ olefin yield.

Although the examples are described herein in terms of isomerizing an iso-olefin to linear olefin, embodiments of the disclosure are applicable to the isomerization of a linear olefin to an iso-olefin.

The particular embodiments disclosed above are merely illustrative, as the present disclosure may be modified and practiced in different but equivalent manners apparent to those skilled in the art having the benefit of the teachings herein. Furthermore, no limitations are intended as to the details of construction or design herein shown, other than as described in the claims below. It is therefore evident that the particular illustrative embodiments disclosed above may be altered of modified and such variations are considered within the scope and spirit of the present disclosure. Alternative embodiments that result from combining, integrating, and/or omitting features of the embodiment(s) are also within the scope of the disclosure.

The following references are incorporated by reference in their entirety for all purposes.
U.S. Pat. No. 3,992,466
U.S. Pat. No. 5,401,704
U.S. Pat. No. 5,648,585
U.S. Pat. No. 6,111,160
U.S. Pat. No. 6,323,384
U.S. Pat. No. 6,652,735
U.S. Pat. No. 9,827,560
“Atlas of Zeolite Structure Types” by W. M. Meier and D. H. Olson, Butterworths, 2nd Edition, 1987

1. A skeletal isomerization process comprising the steps of:
a) feeding, at a weight hourly space velocity (WHSV), a hydrocarbon feed comprising at least one olefin to a reactor containing an isomerization zeolite catalyst, wherein said isomerization zeolite catalyst has a crystallite size that is less than 1 μm in diameter in all directions; and
b) isomerizing said at least one olefin to at least one skeletal isomer product in said reactor for at least one catalyst cycle,
wherein said WHSV is between about 7 to about 30 hr⁻¹.

2. The skeletal isomerization process of claim 1, further comprising the step of recovering said at least one skeletal isomer product from the reactor.

3. The skeletal isomerization process of claim 1, wherein said isomerization zeolite catalyst has a crystallite size of about 0.2 μm in diameter in all directions.

4. The skeletal isomerization process of claim 1, wherein said isomerization zeolite catalyst has a silica to alumina ratio from 10:1 to 60:1.

5. The skeletal isomerization process of claim 1, wherein said isomerization zeolite catalyst additionally comprises a binder material selected from the group consisting of: silica, silica-alumina, bentonite, kaolin, bentonite with alumina, montmorillonite, attapulgite, titania and zirconia.

6. The skeletal isomerization process of claim 1, wherein a temperature of said reactor is from about 340° C. to about 500° C.

7. The skeletal isomerization process of claim 6, wherein a temperature of said reactor is from 380° C. to 425° C.

8. The skeletal isomerization process of claim 1, wherein said at least one olefin is an iso-olefin.

9. The skeletal isomerization process of claim 1, wherein said at least one olefin is a linear olefin.

10. The skeletal isomerization process of claim 1, wherein said at least one olefin is isobutylene and said at least one skeletal isomer product is 1-butene and 2-butene.

11. The skeletal isomerization process of claim 1, wherein said at least one olefin comprises 1-butene and 2-butene, and said at least one skeletal isomer product is isobutylene.

12. The skeletal isomerization process of claim 1, wherein said hydrocarbon feed further comprises alkanes, aromatics, hydrogen and other gases.

13. The skeletal isomerization process of claim 1, wherein said hydrocarbon feed comprises at least 40 wt. % isobutylene.

14. A skeletal isomerization process comprising the steps of:
a) feeding, at a weight hourly space velocity (WHSV) from about 7 to about 30 hr⁻¹, a hydrocarbon feed comprising at least one olefin to a reactor containing an isomerization zeolite catalyst, wherein said isomerization zeolite catalyst has a crystallite size that is less than 1 μm in diameter in all directions; and
b) isomerizing said at least one olefin to at least one skeletal isomer product in said reactor for at least one catalyst cycle,
wherein said catalyst cycle is at least twenty-one days.

15. The skeletal isomerization process of claim 14, wherein said hydrocarbon feed has a weight hourly speed velocity of about 14 hr⁻¹.

16. The skeletal isomerization process of claim 14, wherein said hydrocarbon feed comprises 1-butene and 2-butene, and said at least one skeletal isomer product is isobutylene, or wherein said at least one olefin is isobutylene and said at least one skeletal isomer product is 1-butene and 2-butene.

17. The skeletal isomerization process of claim 14, wherein said catalyst cycle is at least twenty-five days.

18. The skeletal isomerization process of claim 14, wherein said hydrocarbon feed further comprises alkanes, aromatics, hydrogen and other gases.

19. The skeletal isomerization process of claim 14, wherein said hydrocarbon feed comprises at least 40 wt. % isobutylene.

20. A skeletal isomerization process comprising the steps of:
a) feeding, at a weight hourly space velocity (WHSV) from about 7 to about 30 hr⁻¹, a hydrocarbon feed comprising at least one olefin to a reactor at a known temperature and containing an isomerization zeolite catalyst, wherein said isomerization zeolite catalyst has a crystallite size that is less than 1 μm in diameter in all directions; and
b) isomerizing said at least one olefin to at least one skeletal isomer product in said reactor for at least one catalyst cycle,
wherein said catalyst cycle is at least seventeen days;
wherein said known temperature is between about 380° C. and 425° C.
